(19) United States
(12) Patent Application Publication
(10) Pub. No.: US 2022/0084681 A1
(43) Pub. Date: Mar. 17, 2022

(54) METHOD AND APPARATUS FOR PREDICTING MEDICAL EVENT FROM ELECTRONIC MEDICAL RECORD USING PRE_TRAINED ARTFICIAL NEURAL NETWORK

(71) Applicant: VUNO INC., Seoul (KR)

(72) Inventors: Kyungjae CHO, Seongnam-si (KR); Yunseob SHIN, Hwaseong-si (KR); Woong BAE, Seoul (KR)

(73) Assignee: VUNO INC., Seoul (KR)

(21) Appl. No.: 17/474,059

(22) Filed: Sep. 14, 2021

(30) Foreign Application Priority Data
Sep. 15, 2020 (KR) 10-2020-0118663

(51) Int. Cl. G16H 50/30; G16H 50/20; G16H 10/60; G16H 40/67; G06N 3/02; G06N 7/00

(52) U.S. Cl. CPC G16H 50/30; G16H 50/20; G06N 7/005; G16H 40/67; G06N 3/02; G16H 10/60

(57) ABSTRACT

A method of predicting a medical event based on a pre-trained artificial neural network by a computing apparatus, and an apparatus therefor are disclosed. The method includes receiving an electronic medical record vector including a plurality of vital sign components, and outputting the medical event corresponding to the electronic medical record vector using the acritical neural network. The artificial neural network is pre-trained based on learning data, and the learning data includes augmentation electronic medical record vectors which are reconstructed using original electronic medical record vectors pre-acquired at an earlier time point than a first time point based on a mask vector for losing at least one of the plurality of vital sign components of the first time point.

This application claims the benefit of Korean Patent Application No. 10-2020-0118663, filed on Sep. 15, 2020, which is hereby incorporated by reference as if fully set forth herein.

### BACKGROUND OF THE DISCLOSURE

### Field of the Disclosure

The present disclosure relates to a method of predicting a medical event from an electronic medical record using pre-trained artificial neural network, and an apparatus for performing the same.

### Discussion of the Related Art

In a medicine field, electronic medical records are used to predict a medical event of a patient. The electronic medical records are data that records physical changes in a patient over time, and medical personnel including doctors may predict medical events, such as change in the state of disease of a patient or cardiac arrest, from the electronic medical records. However, there are numerous parameters that need to be considered to predict a medical event, and the correlation between the parameters to be considered and medical events is still unclear. In addition, since doctors have different respective clinical experience, the prediction probability of the medical events varies according to the experience of a doctor.

In this context, an artificial neural network has recently been used even in medicine. When it is intended to predict a medical event using the artificial neural network, the artificial neural network may learn using an existing electronic medical record as learning data. The learned artificial neural network may be trained to predict the medical event of a patient based on an electronic medical record of the patient.

In general, ideal electronic medical record data having no loss is used as learning data of the artificial neural network. However, in a general hospital environment, some vital sign components may be omitted from the electronic medical record data according to a time point at which the electronic medical record data is acquired.

Therefore, a learning environment of the artificial neural network may be different from an actual analysis environment because data without loss is used in a learning stage although incomplete data with some loss is input in a process in which the artificial neural network actually predicts the medical event. The difference between the learning environment and the actual analysis environment is problematic in that medical event prediction accuracy of the artificial neural network is lowered.

### SUMMARY OF THE DISCLOSURE

Accordingly, the present disclosure provides a method and apparatus for training an artificial neural network to predict a medical event from an electronic medical record.

The present disclosure provides a method and apparatus for training an artificial neural network capable of more accurately analyzing an electronic medical record collected in a general hospital environment by artificially losing some learning data according to a probability and augmenting the learning data through correction of lost values.

The objects of the present disclosure are not limited to what has been particularly described hereinabove and other objects not described herein will be more clearly understood by persons skilled in the art from the following detailed description of the present disclosure.

According to an aspect, provided herein is a method of predicting a medical event based on a pre-trained artificial neural network by a computing apparatus. The method includes receiving an electronic medical record vector including a plurality of vital sign components, and outputting the medical event corresponding to the electronic medical record vector using the acritical neural network. The artificial neural network is pre-trained based on learning data, and the learning data includes augmentation electronic medical record vectors which are reconstructed using original electronic medical record vectors pre-acquired at an earlier time point than a first time point based on a mask vector for losing at least one of the plurality of vital sign components of the first time point.

The mask vector may include a first mask vector for losing the at least one vital sign component, through masking, which is probabilistically determined based on a first probability vector with respect to a first original electronic medical record vector corresponding to the first time point.

The augmentation electronic medical record vectors may include the first original electronic medical record vector in which the at least one vital sign component lost by the first mask vector is corrected using the pre-acquired original electronic medical record vectors.

The first original electronic medical record vector may be corrected based on an original electronic medical record vector which has a valid value with respect to a vital sign component corresponding to the at least one vital sign component among the pre-acquired original electronic medical record vectors and which is closest to the first time point.

The mask vector may include a second mask vector for losing, through masking, a first original electronic medical record vector at the first time point, determined based on a second probability vector.

The augmentation electronic medical record vectors may include the pre-acquired original electronic medical record vectors shifted in time based on the first time point.

The mask vector may further include a second mask vector for losing, through masking, a second original electronic medical record vector corresponding to a second time point determined based on a second probability vector.

The augmentation electronic medical record vectors may include the first original electronic medical record vector in which the at least one vital sign component lost by the first mask is corrected based on the original electronic medical record vectors pre-acquired at an earlier time point than the first time point, and original electronic medical record vectors pre-acquired at an earlier time point than the second time point shifted in time based on the second time point of the second original electronic medical record vector lost by the second mask vector.

The plural vital sign components may include a heart rate component, a systolic blood pressure component, a diastolic blood pressure component, a respiration rate component, and a body temperature component.

In another aspect, provided herein is a computer-readable storage medium in which a computer program including instructions is recorded. The instructions are configured to cause a computing apparatus to perform receiving an electronic medical record vector including a plurality of vital sign components, and outputting a medical event corresponding to the electronic medical record vector using an acritical neural network. The artificial neural network is pre-trained based on learning data, and the learning data includes augmentation electronic medical record vectors which are reconstructed using original electronic medical record vectors pre-acquired at an earlier time point than a first time point based on a mask vector for losing at least one of the plurality of vital sign components of the first time point.

In another aspect, provided herein is a computing apparatus for predicting a medical event based on a pre-trained artificial neural network. The computing apparatus includes a communicator, and a processor connected to the communicator. The processor is configured to receive an electronic medical record vector including a plurality of vital sign components and output the medical event corresponding to the electronic medical record vector using the acritical neural network. The artificial neural network is pre-trained based on learning data, and the learning data includes augmentation electronic medical record vectors which are reconstructed using original electronic medical record vectors pre-acquired at an earlier time point than a first time point based on a mask vector for losing at least one of the vital sign components of the first time point.

In another aspect, provided herein is a server for predicting a medical event based on a pre-trained artificial neural network. The server includes a processor including one or more cores, a communication interface, and a memory. The processor is configured to receive an electronic medical record vector including a plurality of vital sign components and output the medical event corresponding to the electronic medical record vector using the acritical neural network. The artificial neural network is pre-trained based on learning data, and the learning data includes augmentation electronic medical record vectors which are reconstructed using original electronic medical record vectors pre-acquired at an earlier time point than a first time point based on a mask vector for losing at least one of the vital sign components of the first time point.

It is to be understood that both the foregoing general description and the following detailed description of the present disclosure are exemplary and explanatory and are intended to provide further explanation of the disclosure as claimed.

### BRIEF DESCRIPTION OF THE DRAWINGS

The accompanying drawings, which are included to provide a further understanding of the disclosure and are incorporated in and constitute a part of this application, illustrate embodiment(s) of the disclosure and together with the description serve to explain the principle of the disclosure. In the drawings:

FIG. 1 is a conceptual diagram schematically illustrating an exemplary configuration of a computing apparatus for performing methods described in this disclosure;

FIG. 2 is a flowchart illustrating a machine learning method of an artificial neural network according to an exemplary embodiment;

FIG. 3 is a conceptual diagram exemplarily illustrating a schema of learning data;

FIG. 4 is a flowchart illustrating a process of performing step S120 of FIG. 2 in more detail;

FIG. 5 is a conceptual diagram illustrating a method in which the computing apparatus loses at least a portion of learning data using first mask vectors;

FIG. 6 is a conceptual diagram illustrating a method in which the computing apparatus corrects a portion of learning data lost by first mask vectors;

FIG. 7 is a flowchart illustrating a process of performing step S120 of FIG. 2 in more detail;

FIG. 8 is a conceptual diagram illustrating a method in which the computing apparatus loses at least a portion of learning data using a second mask vector;

FIG. 9 is a conceptual diagram illustrating a method in which the computing apparatus corrects a portion of learning data lost by a second mask vector;

FIG. 10 is a conceptual diagram illustrating a method in which the computing apparatus loses a portion of learning data using a first mask vector and a second mask vector; and

FIG. 11 is a conceptual diagram illustrating a method of correcting an area lost by a first mask vector and a second mask vector.

FIG. 12 is a diagram for explaining a method in which the computing apparatus outputs a medical event using a pre-trained artificial neural network.

### DETAILED DESCRIPTION

In order to clarify the objects, technical solutions, and advantages of the present disclosure, reference will now be made to specific embodiments of the present disclosure, examples of which are illustrated in the accompanying drawings. These embodiments will be described in detail in a way clearly understandable by those of ordinary skill in the art.

An electronic medical record as used throughout the detailed description and claims of this disclosure includes electronically stored medical information of patients or other persons. The medical information may include information about heart rate, blood pressure, respiration rate, body temperature, etc. of a patient or other persons measured at various time points. In the present disclosure, the electronic medical record should be interpreted as comprehensively meaning data obtained by electronically storing biometric information of a patient or other persons, such as an electronic health record (EHR) as well as an electronic medical record (EMR).

Further, the term “training” or “learning” used throughout the detailed description and claims of this disclosure refers to performing machine learning through procedural computing and it will be apparent to those skilled in the art that the term is not intended to refer to a mental action such as an educational activity of a human.

Throughout the detailed description and claims of the present disclosure, the word “include” or “comprise” and variations thereof are not intended to exclude other technical features, additions, components or steps. In addition, “one” or “an” is used to mean more than one, and “another” is defined as at least a second or more.

For persons skilled in the art, other objects, advantages, and features of the present disclosure will be inferred in part from the description and in part from the practice of the present disclosure. The following examples and drawings are provided by way of illustration and not intended to be limiting of the present disclosure. Therefore, the detailed description disclosed herein should not be interpreted as limitative with respect to a specific structure or function and should be interpreted as representing basic data that provides guidelines such that those skilled in the art may variously implement the disclosure as substantially suitable detailed structures.

Further, the present disclosure may include any possible combinations of example embodiments described herein. It should be understood that, although various embodiments differ from each other, they do not need to be exclusive. For example, a specific shape, structure, and feature described herein may be implemented as another example embodiment without departing from the spirit and scope of the present disclosure. In addition, it should be understood that a position or an arrangement of an individual component of each disclosed embodiment may be modified without departing from the spirit and scope of the present disclosure. Accordingly, the following detailed description is not to be construed as being limiting and the scope of the present disclosure, if properly described, is limited by the claims, their equivalents, and all variations within the scope of the claims. In the drawings, like reference numerals refer to the same or like elements throughout various aspects.

Unless the context clearly indicates otherwise, singular forms are intended to include plural forms as well. In the following description of the present disclosure, a detailed description of known functions and configurations incorporated herein will be omitted when it may obscure the subject matter of the present disclosure.

Hereinafter, exemplary embodiments of the present disclosure will be described in detail with reference to the accompanying drawings so that the present disclosure may be easily understood and realized by those skilled in the art

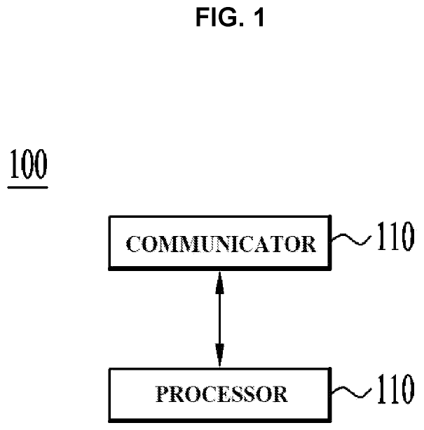

FIG. 1 is a conceptual diagram schematically illustrating an exemplary configuration of a computing apparatus for performing methods described in this disclosure.

A computing apparatus 100 according to an exemplary embodiment may include a communicator 110 and a processor 120 and communicate directly or indirectly with an external computing apparatus (not shown) through the communicator 110. The communicator 110 may correspond to or include a transceiver capable of transmitting and receiving a request and a response to and from another computing apparatus.

Specifically, the computing apparatus 100 may achieve desired system performance using a combination of typical computer hardware (e.g., an apparatus including a computer processor, a memory, a storage, an input device, an output device, components of other existing computing apparatuses, etc.; an electronic communication apparatus such as a router, a switch, etc.; or an electronic information storage system such as a network-attached storage (NAS) and a storage area network (SAN)) and computer software (i.e., instructions that enable a computing apparatus to function in a specific manner).

The communicator 110 of the computing apparatus 100 may transmit and receive a request and a response to and from another computing apparatus interacting therewith. As an example, the request and the response may be implemented using, without being limited to, the same transmission control protocol (TCP) session. For example, the request and the response may be transmitted and received as a user datagram protocol (UDP) datagram. In addition, in a broad sense, the communicator 110 may include a keyboard, a pointing device such as a mouse, and other external input devices for receiving an instruction or a command, and a printer, a display, and other external output devices.

The processor 120 of the computing apparatus 100 may include a hardware configuration, such as a microprocessing unit (MPU), a central processing unit (CPU), a graphics processing unit (GPU), a tensor processing unit (TPU), a cache memory, a data bus, and the like. The processor 120 may further include a software configuration, such as an operating system, an application that performs a specific purpose, and the like. The processor 120 may execute instructions for performing a function of a neural network to be described below.

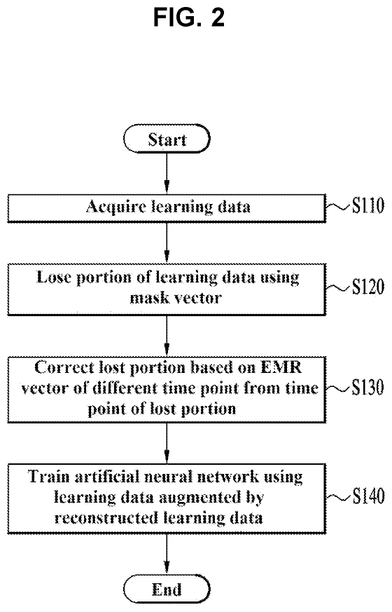

FIG. 2 is a flowchart illustrating a machine learning method of an artificial neural network according to an exemplary embodiment.

Referring to FIG. 2, in step S110, the computing apparatus 100 may acquire learning data. The learning data may be generated based on an EMR. The learning data may include EMR vectors acquired in time series. That is, the learning data may include EMR vectors obtained at a plurality of different time points. Each of the EMR vectors may include vital sign components of a patient or another person obtained at a specific time point. The vital sign components may include, for example, heart rate, systolic blood pressure, diastolic blood pressure, respiration rate, body temperature, etc., but the embodiment is not limited thereto. All parameters measured to obtain biometric information of a patient or another person in a hospital etc. may be included in the vital sign components.

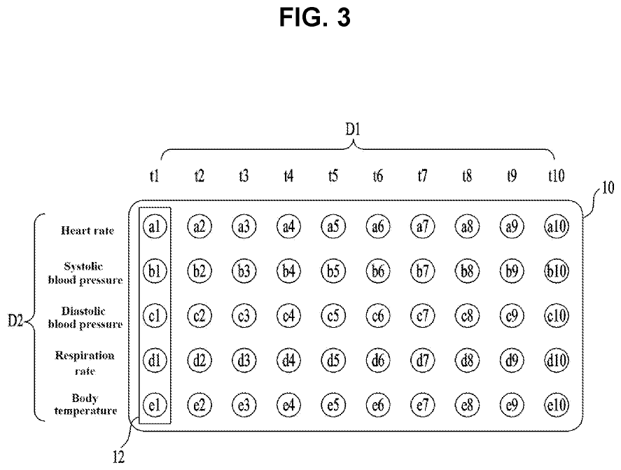

FIG. 3 is a conceptual diagram exemplarily illustrating a schema of learning data 10.

Referring to FIG. 3, the learning data 10 may include EMR vectors 12 obtained at a plurality of time points t1 to t10. Each of the EMR vectors 12 may include vital sign components obtained ata specific time point. Accordingly, the learning data 10 may have a time domain D1 and an vital sign domain D2. The time domain D1 may include the time points t1 to t10 at which the EMR vectors 12 are obtained. The vital sign domain D2 may include vital sign components (e.g., heart rate, systolic blood pressure, diastolic blood pressure, respiration rate, and body temperature) included in each of the EMR vectors 12. For example, in the learning data 10 illustrated in FIG. 3, heart rate acquired at the time point t1 may have a value of a1, and systolic blood pressure acquired at the time point t2 may have a value of b2. The computing apparatus 100 may easily perform a masking operation to be described later by defining the time domain D1 and the vital sign domain D2 in the schema of the learning data 10 as illustrated in FIG. 3.

Referring back to FIG. 2, in step S120, the computing apparatus 100 may lose a portion of the learning data using a mask vector. The mask vector may be used to mask at least a portion of the time domain D1 of the learning data 10 or mask at least a portion of the vital sign domain D2 of the learning data 10. The mask vector may also be used mask at least a portion of each of the time domain D1 and the vital sign domain D2 of the learning data 10. Components masked by the mask vector may be probabilistically determined. Accordingly, whenever the mask vector is newly generated, a portion that the mask vector masks from the learning data 10 may be changed.

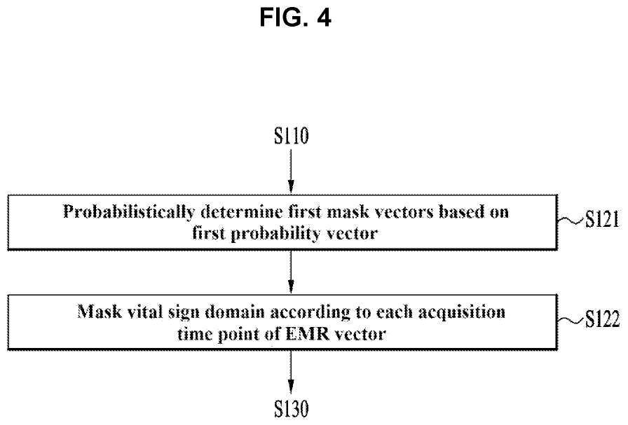

FIG. 4 is a flowchart illustrating a process of performing step S120 of FIG. 2 in more detail.

Referring to FIG. 4, in step S121, the computing apparatus 100 may probabilistically determine first mask vectors for masking the vital sign domain according to each acquisition time point of an EMR vector based on a first probability vector. The computing apparatus 100 may determine the first mask vectors corresponding to respective time points based on a probability vector according to each acquisition time point of the EMR vector. The first mask vectors may be probabilistically determined based on the first probability vector. Accordingly, the types of vital sign components masked by the first mask vectors may vary according to the acquisition time point of the EMR vector.

In step S122, the computing apparatus 100 may mask the vital sign domain according to each acquisition time point of the EMR vector. The computing apparatus 100 may mask the vital sign domain D2 for each acquisition time point of the EMR vectors using the first mask vectors. The computing apparatus 100 may mask the vital sign domain D2 using different first mask vectors with respect to acquisition time points of different EMR vectors.

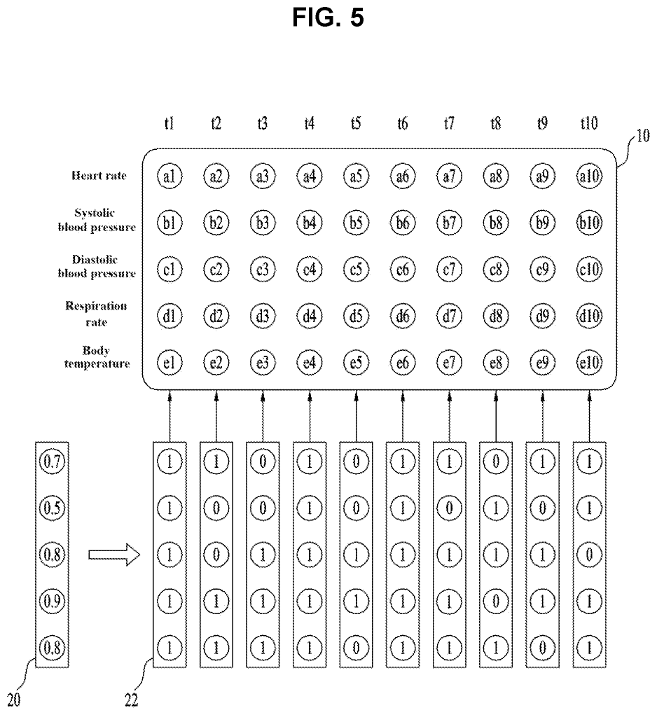

FIG. 5 is a conceptual diagram illustrating a method in which the computing apparatus 100 loses at least a portion of the learning data 10 using first mask vectors 22.

Referring to FIG. 5, the computing apparatus 100 may generate the first mask vectors 22 using a first probability vector 20. The size of the first probability vector may correspond to the size of the vital sign domain D2 of the learning data 10. For example, as illustrated in FIG. 5, when the vital sign domain D2 of the learning data 10 includes five vital sign components, the first probability vector 20 may also include five components.

The components included in the first probability vector 20 may correspond to the vital sign components of the vital sign domain D2 of the learning data 10. For example, the value of the first component of the first probability vector 20 may be a probability of preserving a heart rate component among the vital sign components. That is, the computing apparatus 100 may generate the first mask vectors 22 using the first probability vector 20 such that a probability of losing a heart rate component of EMR vectors obtained at respective time points is 30%. Similarly, the computing apparatus 100 may generate the first mask vectors 22 using the first probability vector 20 such that a probability of losing a systolic blood pressure component of the EMR vectors obtained at respective time points is 50%.

Each component of the first mask vectors 22 may have a binary value. In the first mask vectors 22, a value of “1” indicates that data of a corresponding portion is preserved during masking, and a value of “0” indicates that data of a corresponding portion is lost during masking. In FIG. 5, the binarization notation method is indicated by “1” and “0”, but this is only an example for explaining the embodiment and the binarization notation method may be changed in other ways.

The computing apparatus 100 may obtain the first mask vectors 22 based on the first probability vector 20 to mask the vital sign domain D2 at each of the time points t1 to t10 at which the EMR vectors are obtained. For example, all components of the first mask vector 22 for masking the EMR vectors obtained at the time point t1 may have values of “1”. Accordingly, values of the EMR vectors obtained at the time point t1 may all be preserved even after masking is performed. On the other hand, the second and third components of the first mask vector 22 for masking the EMR vectors obtained at the time point t2 may have values of “0”. Therefore, a value of b2 and a value of c2 corresponding to systolic blood pressure and diastolic blood pressure) n the EMR vectors obtained at the time point t2 may be lost by masking.

As described above, since the computing apparatus 100 probabilistically generates the first mask vector 22 based on the first probability vector 20 at each time point, the types of components lost at each time point may be probabilistically determined. Since a lost portion of the learning data 10 is probabilistically determined in the vital sign domain at each acquisition time of the EMR vectors, this may cause a result similar to omission of some vital sign components at each acquisition time point of an EMR in an actual hospital environment.

Referring back to FIG. 2, in step S130, the computing apparatus 100 may correct a lost portion of the learning data 100 based on an EMR vector obtained at a different time point from a time point of the lost portion. The computing apparatus 100 may reconstruct the learning data 10 by correcting the lost portion.

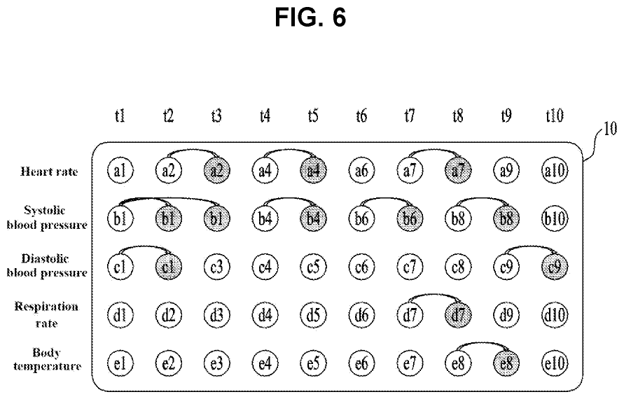

FIG. 6 is a conceptual diagram illustrating a method in which the computing apparatus 100 corrects a portion of the learning data 10 lost by the first mask vectors 22.

Referring to FIG. 6, the computing apparatus 100 may correct the lost portion based on an EMR vector obtained at an earlier time point than a time point of the lost portion of the learning data 10. For example, the computing apparatus 100 may correct the values of b2 and c2 lost at the time point t2 using values of b1 and c1 of an EMR vector obtained at the time point t1 earlier than the time point t2. The computing apparatus 100 may copy the value of b1, which is systolic blood pressure of the time point t1, and store the value of b1 as systolic blood pressure value at the time point t2. Similarly, the computing apparatus 100 may copy the value of c1, which is diastolic blood pressure of the time point t1, and store the value of c1 as a diastolic blood pressure value at the time point t2.

The computing apparatus 100 may correct the lost portion with reference to a second EMR vector having a valid value for the lost portion, obtained at a previous time point closest to an acquisition time point of a first EMR vector including the lost portion. For example, the computing apparatus 100 may correct a heart rate component lost at the time point t3 by copying a value of a2, which is the heart rate component of the time point t2 closest to the time point t3 at which the heart rate component is lost. In addition, since a systolic blood pressure component of the time point t2 closest to the time point t3 is also lost, the computing apparatus 100 may correct the systolic blood pressure component lost at the time point t3 by copying a value of b1, which is a systolic blood pressure component of the time point t1.

Referring back to FIG. 2, in step S140, the computing apparatus 100 may augment learning data by adding learning data reconstructed through correction of the lost portion of the learning data 10 to existing learning data. By probabilistically reconstructing and augmenting the learning data, the computing apparatus 100 may implement an artificial neural network capable of effectively operating in a hospital environment in which vital sign data may be lost. In addition, the computing apparatus 100 may correct the lost portion with reference to an EMR vector at a different time point in a reconstruction process of the learning data, so that the artificial neural network may effectively operate even if the lost portion is corrected in the same way in actual analysis data.

Hereinabove, an example of reconstructing and augmenting the learning data using the first mask vectors for the vital sign domain of the learning data has been described. However, the embodiment is not limited thereto and a method of losing a portion of the learning data may be changed in various ways. For example, the computing apparatus 100 may lose a portion of the learning data using a second mask vector for the time domain of the learning data.

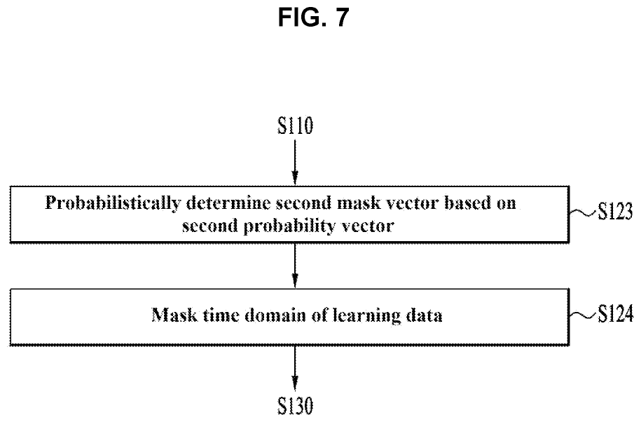

FIG. 7 is a flowchart illustrating a process of performing step S120 of FIG. 2 in more detail.

Referring to FIG. 7, in step S123, the computing apparatus 100 may probabilistically determine a second mask vector for masking the time domain based on a second probability vector.

In step S124, the computing apparatus 100 may perform masking on the time domain of the learning data 10 using the second mask vector. The computing apparatus 100 may lose EMR vectors obtained at least a portion of the time points t1 to t10 included in the time domain.

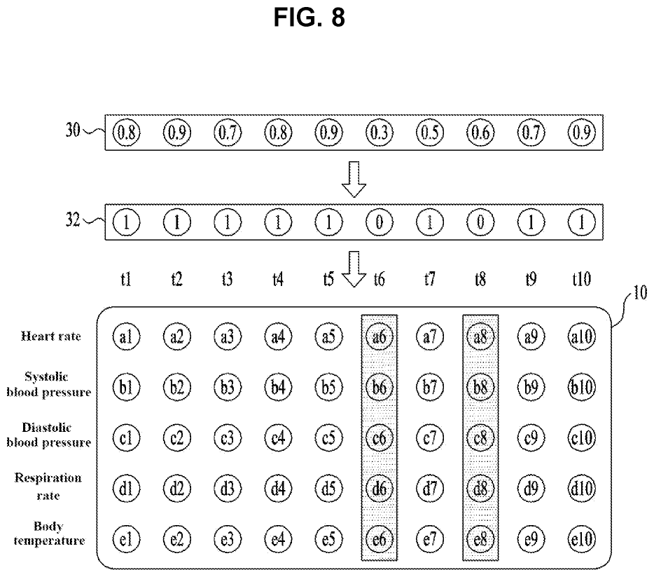

FIG. 8 is a conceptual diagram illustrating a method in which the computing apparatus 100 loses at least a portion of the learning data 10 using a second mask vector.

Referring to FIG. 8, the computing apparatus 100 may generate a second mask vector 32 using a second probability vector 30. The size of the second probability vector 30 may correspond to the size of the time domain D1 of the learning data 10. For example, as illustrated in FIG. 8, when the time domain D1 of the learning data 10 includes 10 time points t1 to t10, the second probability vector 20 may also include 10 components.

Components included in the second probability vector 30 may correspond to acquisition time points t1 to t10 of the EMR vectors included in the time domain D1 of the learning data 10. For example, the first component value of the second probability vector 30 may be a probability of preserving the EMR vector obtained at the time point t1. According to the embodiment illustrated in FIG. 8, the computing apparatus 100 may generate the second mask vector 32 using the second probability vector 30 such that a probability of losing an EMR vector obtained at the time point t1 is 20% and a probability of losing an EMR vector obtained at the time point t2 is 10%.

Like the first mask vector 22, the second mask vector 32 may have binary values. The computing apparatus 100 may probabilistically determine components of the second mask vector 32 using the second probability vector 30. For example, since a value of the first component of the second mask vector 32 illustrated in FIG. 8 is “1”, the computing apparatus 100 may preserve an EMR vector obtained at the time point t1. On the other hand, since the sixth component value of the second mask vector 32 is “0”, the computing apparatus 100 may lose an EMR vector obtained at the time point t6. Since the second mask vector 32 is probabilistically generated, a time point at which an EMR vector is lost may also be probabilistically determined.

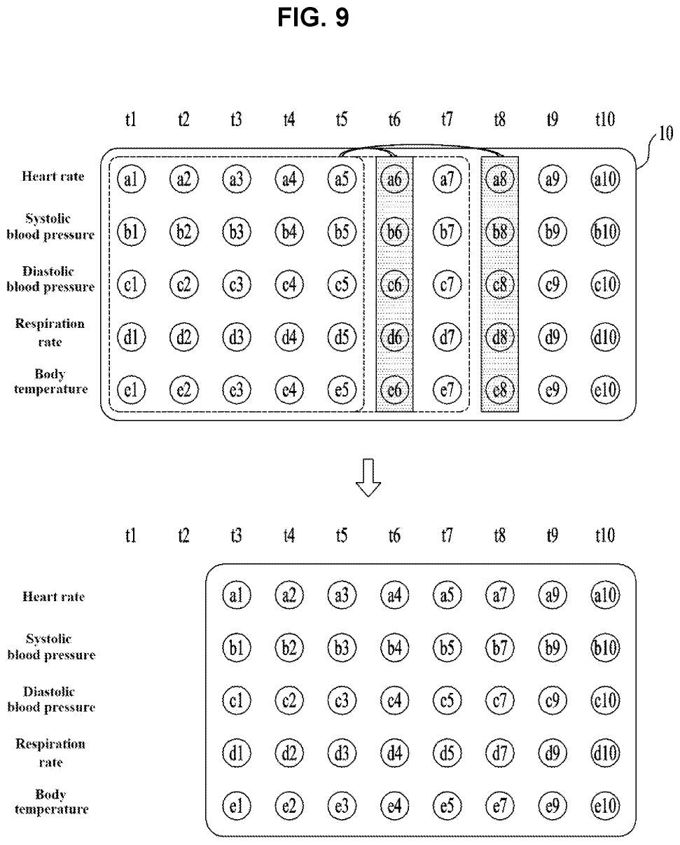

FIG. 9 is a conceptual diagram illustrating a method in which the computing apparatus 100 corrects a portion of the learning data 10 lost by the second mask vector 32.

Referring to FIG. 9, the computing apparatus 100 may correct a lost portion by shifting, in the time domain, EMR vectors obtained at earlier time points than an acquisition time point of an EMR vector lost by the second mask vector 32. For example, an EMR vector acquired at the time point t6 may be lost by the second mask vector 32. The computing apparatus 100 may correct a lost area generated at the time point t6 by shifting EMR vectors acquired at the time points t1 to t5 in the time domain. In addition, the computing apparatus 100 may correct a lost area generated at the time point t8 by shifting EMR vectors existing at the time points t1 to t7 in the time domain.

The computing apparatus 100 may reconstruct the learning data 10 by correcting the lost area. The computing apparatus 100 may augment the learning data by adding the reconstructed learning data to the existing learning data. The computing apparatus 100 may implement an artificial neural network capable of effectively operating in a hospital environment in which an EMR may be omitted at some time points by probabilistically losing an EMR vector of a specific time point and reconstructing and augmenting the learning data. In addition, the computing apparatus 100 corrects the lost portion by shifting EMR vectors of earlier time points than a time point of the lost portion in the time domain, so that the artificial neural network may effectively operate even if the lost portion is corrected in the same way in actual analysis data.

Hereinabove, only the case of using any one of the first mask vector 22 and the second mask vector 32 has been described, but the embodiment is not limited thereto. For example, the computing apparatus 100 may lose at least a portion of the learning data using both the first mask vector 22 and the second mask vector 32.

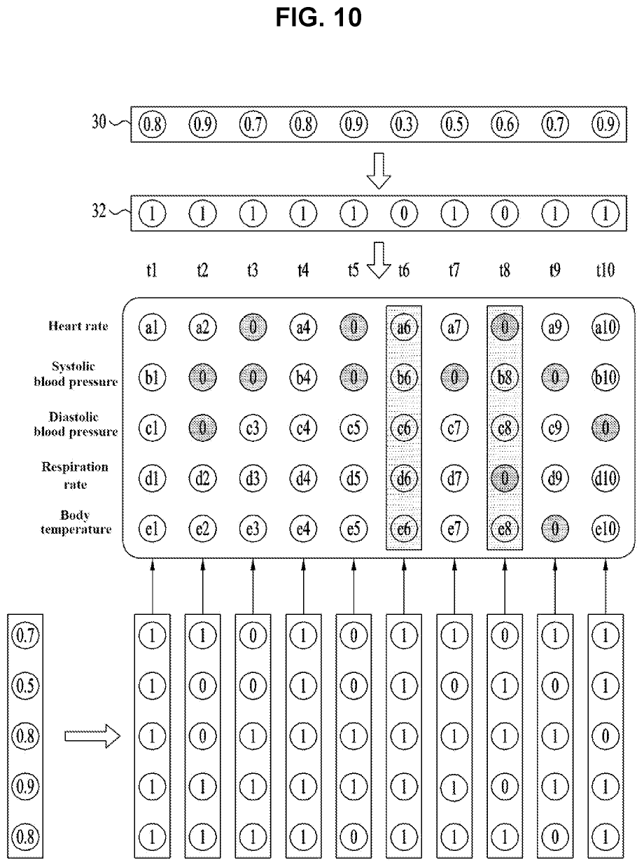

FIG. 10 is a conceptual diagram illustrating a method in which the computing apparatus 100 loses a portion of the learning data using the first mask vector 22 and the second mask vector 32.

Referring to FIG. 10, the computing apparatus 100 may lose some vital signal components by probabilistically performing masking on the vital sign domain at each acquisition time point of an EMR vector using the first mask vector 22. The computing apparatus 100 may lose EMR vectors acquired at some time points by probabilistically performing masking on the time domain using the second mask vector 32.

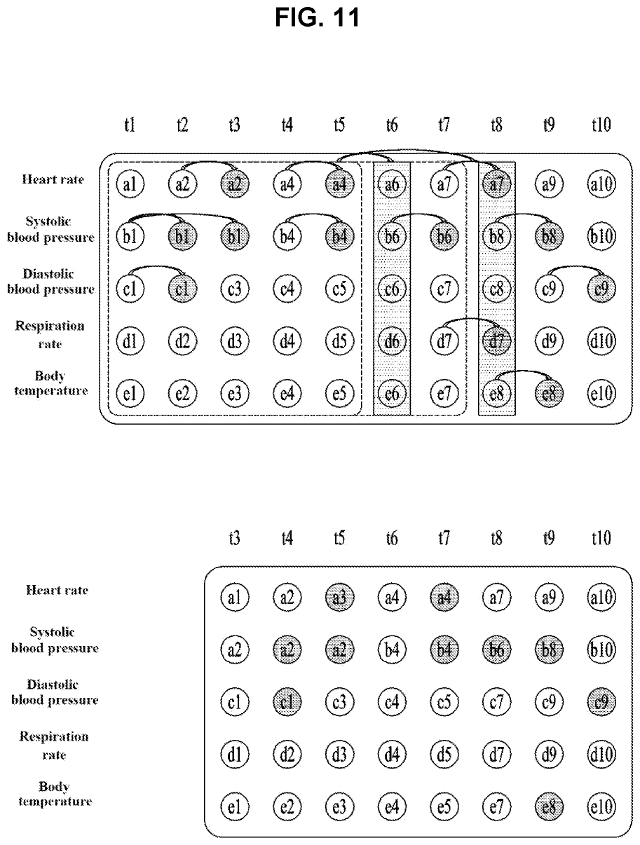

FIG. 11 is a conceptual diagram illustrating a method of correcting an area lost by the first mask vector 22 and the second mask vector 32.

Referring to FIG. 11, the computing apparatus 100 may correct a lost area by copying a vital sign component of an EMR vector acquired at an earlier time point than a time point of an area lost by the first mask vector 22. The computing apparatus 100 may correct a lost portion by shifting, in the time domain, EMR vectors of earlier time points than a time point of a portion lost by the second mask vector 32. The computing apparatus 100 may augment the learning data using the reconstructed learning data. The computing apparatus 100 may train the artificial neural network using the augmented learning data.

As described above, the EMR vectors obtained in time series may be used as EMR vectors for pre-training the artificial neural network. Hereinbelow, in order to distinguish between EMR vectors obtained in time series and EMR vectors from which an actual medical event is predicted, a description will be given by defining EMR vectors related to learning data as original EMR vectors.

In this case, the augmented learning data may include augmentation EMR vectors, which are the reconstructed original EMR vectors. The artificial neural network performs pre-training based on the augmented learning data, thereby achieving robustness over the case in which at least one vital sign component included in actual EMR vectors (or EMR vectors) or a part of the actual EMR vectors is lost.

Hereinafter, a method in which the computing apparatus outputs a medical event using a pre-trained artificial neural network according to the above-described pre-training will be described in detail.

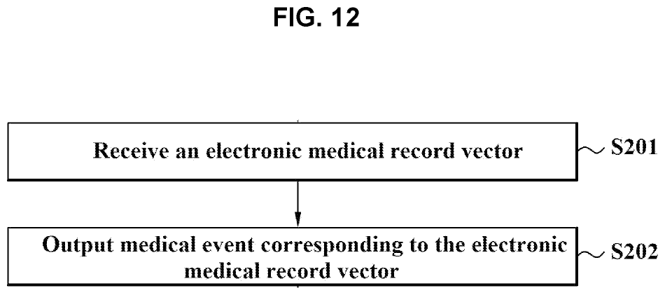

FIG. 12 is a diagram for explaining a method in which the computing apparatus outputs a medical event using a pre-trained artificial neural network.

Referring to FIG. 12, the computing apparatus may receive at least one EMR vector including a plurality of vital sign components (S201). The computing apparatus may determine or predict a corresponding medical event based on the vital sign components included in the at least one EMR vector using the pre-trained artificial neural network.

Next, the computing apparatus may output a medical event corresponding to the EMR vector using the pre-trained artificial neural network (S202).

The pre-trained artificial neural network may be pre-trained based on learning data (or augmented learning data) including augmentation EMR vectors reconstructed by correcting partially lost original EMR vectors as described with reference to FIGS. 1 to 11. As such, the artificial neural network is pre-trained through training based on the augmentation EMR vectors, so that the artificial neural network may more accurately predict or determine a corresponding medical event in an effective manner even if some of the vital sign components included in the received EMR vectors are lost or some of the EMR vectors received in time series are lost and corrected.

Specifically, the pre-trained artificial neural network may be pre-trained based on the learning data including the augmentation EMR vectors as described with reference to FIGS. 1 to 11. As described previously, the augmentation EMR vectors may be a plurality of reconstructed original EMR vectors. The reconstructed original EMR vectors may be original EMR vectors in which partial vital sign components (and/or partial original EMR vectors) are lost by the first mask vector and/or the second mask vector and the lost partial vital sign components (and/or partial original EMR vectors) are corrected.

Specifically, at least one of a plurality of vital sign components included in an original EMR vector corresponding to a first time point may be masked by the first mask vector. In other words, the at least one vital sign component may be masked by the first mask vector so that the at least one vital sign component may be lost from the original EMR vector corresponding to the first time point. Here, the at least one vital sign component which is lost through masking by the first mask vector may be probabilistically determined by the first probability vector. The at least one lost vital sign component may be corrected based on original EMR vectors which are pre-acquired at an earlier time point than the first time point, so that the original EMR vector corresponding to the first time point may be reconstructed as the augmentation EMR vector.

For example, the original EMR vector corresponding to the first time point may be corrected based on an original EMR vector, which has a valid value with respect to a vital sign component corresponding to the at least one vital sign component and is closest to the first time point, among the pre-acquired original EMR vectors. Meanwhile, an original EMR vector from which partial vital sign components are lost by the first mask vector may be defined as a first original EMR vector.

In addition, the learning data may include, as described with reference to FIGS. 1 to 11, corrected original EMRs (or augmentation EMRs) obtained by correcting the original EMR vector of the first time point (and/or a second time point) lost by the second mask vector. The first time point (and/or the second time point) at which masking is to be performed among a plurality of acquisition time points corresponding respectively to multiple original EMR vectors may be probabilistically determined based on the second probability vector. The original EMR vector (or a second EMR vector) corresponding to the first time point (and/or second time point) may be masked and lost using the second mask vector. The multiple original EMR vectors may be reconstructed as the augmented EMR vectors such a manner that original EMR vectors pre-acquired at an earlier time point than the first time point are shifted in time based on the first time point. The first time point may include at least one time point, and the second mask vector may lose at least one of the multiple original EMRs through masking.

For example, as illustrated in FIG. 9, the original EMR vectors may be reconstructed such a manner that pre-acquired original EMRs closest to the first time point are shifted to the first time point and the next closest pre-acquired original EMRs are sequentially shifted to a time point (i.e., acquisition time point) corresponding to a time point before the closest pre-acquired original EMRs are shifted. Alternatively, an original EMR vector lost by the second mask vector may be defined as a second original EMR vector.

In this way, the artificial neural network may be pre-trained based on learning data including the reconfigured original EMR vectors (or the augmentation EMR vectors). In this case, the artificial neural network may more accurately predict corresponding medical events even if a part of the received EMR is corrected due to loss.

The method and apparatus for training the artificial neural network according to exemplary embodiments have been described hereinabove with reference to FIGS. 1 to 12. According to at least one embodiment, the computing apparatus 100 may learn the artificial neural network so as to have robustness against data loss by losing a portion of the learning data using a mask vector generated based on a probability. According to at least one embodiment, the computing apparatus 100 may reconstruct the learning data using the first mask vector for the vital sign domain of the learning data, so that a probability of omitting some vital sign components at an acquisition time point of an EMR in a hospital environment may be reflected in the learning data. According to at least one embodiment, the computing apparatus 100 may reconstruct the learning data using the second mask vector for the time domain of the learning data, so that a probability of omitting some vital sign components at an acquisition time of an EMR in a hospital environment may be reflected in the learning data. According to at least one embodiment, the computing apparatus may correct a lost portion with reference to an EMR vector of a different time point from a time point of the lost portion in the reconstruction process of the learning data, so that the artificial neural network may effectively operate even if the lost portion is corrected in the same way in actual analysis data. According to at least one embodiment, since various mask vectors may be generated by a probability vector, the computing apparatus may easily augment the learning data in large amounts.

Those skilled in the art may easily understand that the methods and/or processes and steps thereof described in the above embodiments may be implemented using hardware, software, or a combination of hardware and software suitable for a specific usage. Hardware may include a general-purpose computer and/or an exclusive computing apparatus, a specific computing apparatus, or a special feature or component of the specific computing apparatus. The processes may be implemented using at least one microprocessor, microcontroller, embedded microcontroller, programmable digital signal processor, or programmable device, having an internal and/or external memory. In addition, or, as an alternative, the processes may be implemented using an application specific integrated circuit (ASIC), a programmable gate array, a programmable array logic (PAL), or an arbitrary device configured to process electronic signals, or a combination thereof. Targets of technical solutions of the present disclosure or portions contributing to the prior art may be configured in a form of program instructions performed by various computer components and may be stored in machine-readable recording media. The machine-readable recording media may include, alone or in combination, program instructions, data files, data structures, and the like. The program instructions recorded in the machine-readable recording media may be specially designed and configured for the present disclosure or may be known to those skilled in the art of computer software. Examples of the media may include magnetic media such as hard disks, floppy disks, and magnetic tapes; optical media such as CD-ROM discs, DVDs, and Blu-ray; magneto-optical media such as floptical disks; and hardware devices that are specially configured to store and perform program instructions, such as a ROM, a RAM, a flash memory, and the like. The program instructions may be produced by structural programming languages such as C, object-oriented programming languages such as C++, or high or low-level programming languages (assembly languages, hardware technical languages, database programming languages and techniques), which are capable of being stored, compiled, or interpreted in order to run not only on one of the aforementioned devices but also on a processor, a processor architecture or a heterogeneous combination of different hardware and software combinations, or a machine capable of executing any other program instructions. The examples of the program instructions include machine language code, byte code, and high-level language code executable by a computer using an interpreter etc.

Therefore, according to aspect of the present disclosure, the aforementioned methods and combinations thereof may be implemented by one or more computing apparatuses as executable code that performs the respective steps. According to another aspect, the methods may be implemented by systems that perform the steps and may be distributed over a plurality of devices in various manners or all of the functions may be integrated into a single exclusive, stand-alone device, or different hardware. According to still another aspect, devices that perform steps associated with the aforementioned processes may include the aforementioned hardware and/or software. All of the sequences and combinations associated with the processes are to be included in the scope of the present disclosure.

For example, the described hardware devices may be configured to act as one or more software modules in order to perform the operations of the present disclosure, or vice versa. The hardware devices may include a processor, such as an MPU, a CPU, a GPU, and a TPU, configured to be combined with a memory such as ROM/RAM for storing program instructions and to execute the instructions stored in the memory, and may include a communicator capable of transmitting and receiving a signal to and from an external device. In addition, the hardware devices may include a keyboard, a mouse, and an external input device for receiving instructions created by developers.

According to at least one embodiment, the computing apparatus may train an artificial neural network to have robustness against data loss by losing a portion of learning data using a mask vector generated based on a probability.

According to at least one embodiment, the computing apparatus may reconstruct learning data using a first mask vector for a vital sign domain of learning data, so that a possibility that some vital sign components may be omitted at each acquisition time point of an electronic medical record in a hospital environment may be reflected in the learning data.

According to at least one embodiment, the computing apparatus may reconstruct learning data using a second mask vector for a time domain of the learning data, so that a possibility that an electronic medical record at a specific time point may be omitted in a hospital environment may be reflected in the learning data.

According to at least one embodiment, the computing apparatus may correct a lost part of learning data with reference to an electronic medical record vector at a different point in a reconstruction process of the learning data, so that an artificial neural network may effectively operate even if the lost part is corrected in the same way in actual analysis data.

According to at least one embodiment, since various mask vectors may be generated by a probability vector, the computing apparatus may easily augment learning data in large amounts.

While the present disclosure is described with reference to specific matters such as components, some example embodiments, and drawings, they are merely provided to aid in general understanding of the present disclosure and this disclosure is not limited to the example embodiments. It will be apparent to those skilled in the art that various alternations and modifications in form and detail may be made from the present disclosure.

Therefore, the scope of the present disclosure is not defined by the above-described embodiments but by the claims and their equivalents, and all variations within the scope of the claims and their equivalents are to be construed as being included in the disclosure.

Such equally or equivalently modified examples may include, for example, logically equivalent methods capable of achieving the same results as those acquired by implementing the method according to this disclosure. Accordingly, the spirit and scope of the present disclosure are not limited to the aforementioned examples and should be understood as having the broadest meaning allowable by law.

What is claimed is:

1. A method of predicting a medical event based on a pre-trained artificial neural network by a computing apparatus, the method comprising:
receiving an electronic medical record vector including a plurality of vital sign components; and
outputting the medical event corresponding to the electronic medical record vector using the acritical neural network,
wherein the artificial neural network is pre-trained based on learning data, and
wherein the learning data includes augmentation electronic medical record vectors which are reconstructed using original electronic medical record vectors pre-acquired at an earlier time point than a first time point based on a mask vector for losing at least one of the plurality of vital sign components of the first time point.

2. The method of claim 1, wherein the mask vector includes a first mask vector for losing the at least one vital sign component which is probabilistically determined based on a first probability vector, through masking, with respect to a first original electronic medical record vector corresponding to the first time point.

3. The method of claim 2, wherein the augmentation electronic medical record vectors include the first original electronic medical record vector in which the at least one vital sign component lost by the first mask vector is corrected using the pre-acquired original electronic medical record vectors.

4. The method of claim 3, wherein the first original electronic medical record vector is corrected based on an original electronic medical record vector which has a valid value with respect to a vital sign component corresponding to the at least one vital sign component among the pre-acquired original electronic medical record vectors and which is closest to the first time point.

5. The method of claim 1, wherein the mask vector includes a second mask vector for losing, through masking, a first original electronic medical record vector at the first time point, determined based on a second probability vector.

6. The method of claim 5, wherein the augmentation electronic medical record vectors include the pre-acquired original electronic medical record vectors shifted in time based on the first time point.

7. The method of claim 2, wherein the mask vector further includes a second mask vector for losing, through masking, a second original electronic medical record vector corresponding to a second time point determined based on a second probability vector.

8. The method of claim 7, wherein the augmentation electronic medical record vectors include:
the first original electronic medical record vector in which the at least one vital sign component lost by the first mask is corrected based on the original electronic medical record vectors pre-acquired at an earlier time point than the first time point, and
original electronic medical record vectors pre-acquired at an earlier time point than the second time point shifted in time based on the second time point of the second original electronic medical record vector lost by the second mask vector.

9. The method of claim 1, wherein the plurality of vital sign components include a heart rate component, a systolic blood pressure component, a diastolic blood pressure component, a respiration rate component, and a body temperature component.

10. A computing apparatus for predicting a medical event based on a pre-trained artificial neural network, the computing apparatus comprising:
a communicator; and
a processor connected to the communicator,
wherein the processor is configured to receive an electronic medical record vector including a plurality of vital sign components and output the medical event corresponding to the electronic medical record vector using the acritical neural network,
wherein the artificial neural network is pre-trained based on learning data, and
wherein the learning data includes augmentation electronic medical record vectors which are reconstructed using original electronic medical record vectors pre-acquired at an earlier time point than a first time point based on a mask vector for losing at least one of the plurality of vital sign components of the first time point.

11. The computing apparatus of claim 10, wherein the mask vector includes a first mask vector for losing the at least one vital sign component which is probabilistically determined based on a first probability vector, through masking, with respect to a first original electronic medical record vector corresponding to the first time point.

12. The computing apparatus of claim 11, wherein the augmentation electronic medical record vectors include the first original electronic medical record vector in which the at least one vital sign component lost by the first mask vector is corrected using the pre-acquired original electronic medical record vectors.

13. The computing apparatus of claim 12, wherein the first original electronic medical record vector is corrected based on an original electronic medical record vector which has a valid value with respect to a vital sign component corresponding to the at least one vital sign component among the pre-acquired original electronic medical record vectors and which is closest to the first time point.

14. The computing apparatus of claim 10, wherein the mask vector includes a second mask vector for losing, through masking, a first original electronic medical record vector at the first time point, determined based on a second probability vector.

15. The computing apparatus of claim 14, wherein the augmentation electronic medical record vectors include the pre-acquired original electronic medical record vectors shifted in time based on the first time point.

16. The computing apparatus of claim 11, wherein the mask vector further includes a second mask vector for losing, through masking, a second original electronic medical record vector corresponding to a second time point determined based on a second probability vector.

17. The computing apparatus of claim 16 wherein the augmentation electronic medical record vectors include:
the first original electronic medical record vector in which the at least one vital sign component lost by the first mask is corrected based on the original electronic medical record vectors pre-acquired at an earlier time point than the first time point, and
original electronic medical record vectors pre-acquired at an earlier time point than the second time point shifted in time based on the second time point of the second original electronic medical record vector lost by the second mask vector.

18. The computing apparatus of claim 10, wherein the plurality of vital sign components include a heart rate component, a systolic blood pressure component, a diastolic blood pressure component, a respiration rate component, and a body temperature component.

19. A server for predicting a medical event based on a pre-trained artificial neural network, the server comprising:
a processor including one or more cores;
a communication interface; and
a memory,
wherein the processor is configured to receive an electronic medical record vector including a plurality of vital sign components and output the medical event corresponding to the electronic medical record vector using the acritical neural network,
wherein the artificial neural network is pre-trained based on learning data, and
wherein the learning data includes augmentation electronic medical record vectors which are reconstructed using original electronic medical record vectors pre-acquired at an earlier time point than a first time point based on a mask vector for losing at least one of the plurality of vital sign components of the first time point.
